(19) United States
(12) Patent Application Publication
(10) Pub. No.: US 2021/0113495 A1
(43) Pub. Date: Apr. 22, 2021

(54) METHOD OF TREATING EPILEPSY OR EPILEPTIC ENCEPHALOPATHY WITH FENFLURAMINE IN PATIENTS WITHOUT PULMONARY HYPERTENSION

(71) Applicant: ZOGENIX INTERNATIONAL LIMITED, Berkshire (GB)

(72) Inventors: Brooks M. BOYD, Emeryville, CA (US); Gail FARFEL, Emeryville, CA (US)

(73) Assignee: ZOGENIX INTERNATIONAL LIMITED, Berkshire (GB)

(21) Appl. No.: 17/253,545

(22) PCT Filed: Jul. 3, 2019
(86) PCT No.: PCT/US2019/040586

§ 371 Date: Dec. 17, 2020

Related U.S. Application Data
(60) Provisional application No. 62/696,153, filed on Jul. 10, 2018.
(60) Provisional application No. 62/699,987, filed on Jul. 18, 2018.

(51) Int. Cl. A61K 31/137; A61K 31/341; A61K 31/585; A61K 31/4965; A61K 31/343; A61K 31/505; A61K 31/4985; A61K 31/519; A61K 31/191; A61K 31/20; A61K 31/192; A61K 9/00; A61P 25/08; A61P 9/12

(52) U.S. Cl. CPC A61K 31/137; A61K 31/341; A61K 31/585; A61K 31/4965; A61K 31/343; A61K 31/505; A61P 9/12; A61K 31/519; A61K 31/191; A61K 31/20; A61K 31/192; A61K 9/0053; A61P 25/08; A61K 31/4985

(57) ABSTRACT

Provided herein are methods of identifying a patient population as candidates for effective treatment of symptoms of epilepsy or epileptic encephalopathy with fenfluramine or a pharmaceutically acceptable salt thereof, by pre-selecting patients diagnosed with epilepsy or epileptic encephalopathy, assessing the pre-selected patients for being at risk of or exhibiting symptoms of pulmonary hypertension, treating and re-assessing the selected and treated patients for pulmonary hypertension, and identifying treated and re-assessed patients not exhibiting pulmonary hypertension as candidates for effective treatment of epilepsy or epileptic encephalopathy with fenfluramine or a pharmaceutically acceptable salt thereof.

### FIELD

This disclosure relates generally to methods of identifying a patient population as candidates for effective treatment of symptoms of epilepsy or epileptic encephalopathy with fenfluramine or a pharmaceutically acceptable salt thereof.

### INTRODUCTION

Pulmonary hypertension (PH or PHTN; also known as “idiopathic pulmonary arterial hypertension” (IPAH)) is a condition of increased blood pressure within the arteries of the lungs. Symptoms include shortness of breath, syncope (fainting), tiredness, chest pain, swelling of the legs, and a fast heartbeat. PH is a devastating and highly morbid disease characterized by progressive obliteration of precapillary arterioles. The underlying mechanism typically involves inflammation of the arteries in the lungs. PH can be divided into five major types: “arterial plexiform,” “veno-occlusive,” “hypoxic,” “thromboembolic” and “unclear multifactorial” varieties, and a series of tests (physical examination and detailed family history, echocardiography, ventilation/perfusion scintigraphy, right heart catheterization, CT scan, etc.) must be performed to distinguish/diagnose the type of PH.

Pulmonary arterial hypertension (PAH) is diagnosed only after exclusion of other possible causes of pulmonary hypertension. Further description of the diagnosis of pulmonary hypertension, including diagnosis by defined by ranges of medical test findings, can be found in, for example, Hoeper M M, et al. (December 2013) Journal of the American College of Cardiology. 62 (25 Suppl): D42-50. The prognosis for PAH has an untreated median survival of 2-3 years from time of diagnosis, with the cause of death usually being right ventricular failure (cor pulmonale). However, a recent study of patients who had started treatment with bosentan (Tracleer) showed that 89% patients were alive at 2 years, and with new therapies, survival rates are increasing. For 2,635 patients enrolled in The Registry to Evaluate Early and Long-term Pulmonary Arterial Hypertension Disease Management (REVEAL Registry) from March 2006 to December 2009, 1-, 3-, 5-, and 7-year survival rates were 85%, 68%, 57%, and 49%, respectively. For patients with idiopathic/familial PAH, survival rates were 91%, 74%, 65%, and 59%. However, levels of mortality remain very high in pregnant women with severe pulmonary arterial hypertension.

A number of supportive measures such as oxygen therapy, diuretics, and medications to inhibit clotting may be used to treat PH. Treatment is often directed to optimize left ventricular function with the use of diuretics, digoxins, blood thinners, or to repair/replace the mitral valve or aortic valve. Specific medications for PH include epoprostenol, treprostinil, iloprost, bosentan, ambrisentan, macitentan, and sildenafil. Surgical options, including a lung transplant may be recommended in certain cases. For example, atrial septostomy is a surgical procedure that creates a communication between the right and left atria; this relieves pressure on the right side of the heart, but at the cost of lower oxygen levels in blood (hypoxia). Lung transplantation cures pulmonary arterial hypertension, but leaves the patient with the complications of transplantation, and a post-surgical median survival of just over five years.

Not all risk factors for PH are known, but some include a family history of PH, genetic predisposition, prior blood clots in the lungs/pulmonary embolism, HIV/AIDS, sickle cell disease, cocaine use, chronic obstructive pulmonary disease (COPD), sleep apnea, living at high altitudes, and problems with the mitral valve. Appetite suppressants, including dexfenfluramine, also have been reported to precipitate or hasten the development of PH. (Brenot, et al., (1993) Br. Heart J. 70(6):537-541). Other risk factors of PAH include primary pulmonary hypertension, heart failure, left ventricular failure, valvular heart disease; early/initial symptoms of PH include exertional dyspnea, lethargy, fatigue (alone or in combination with other symptoms and/or risk factors); and late symptoms include exertional chest pain, angina, exertional syncope, peripheral edema, anorexia, abdominal pain (alone or in combination with other symptoms and/or risk factors).

Despite past cardiovascular safety concerns that arose when high doses of fenfluramine were used for treatment of adult obesity, other therapeutic uses for that product that outweigh the known risks of fenfluramine have been identified and used to therapeutic benefit. New treatment options are sorely needed for epilepsy or epileptic encephalopathy, and in particular, epilepsy syndromes which are refractory to known treatments. Epilepsy is a condition of the brain marked by a susceptibility to recurrent seizures. Historically, investigation of fenfluramine's efficacy in epilepsy patients, while showing some initial promise, was far from definitive, and shared a common paradigm, i.e., that fenfluramine's primary effects were on behaviors that caused or induced seizures, not treating or preventing the seizure itself.

Certain forms of epilepsy and epileptic encephalopathy have been successfully treated with fenfluramine. However, treatment of symptoms of epilepsy of epileptic encephalopathy with fenfluramine may be problematic and contraindicated in cases in which the subject to be treated has pulmonary hypertension.

A need remains for effective treatment of epilepsy or epileptic encephalopathy with fenfluramine. The present disclosure relates generally to methods of identifying a population of patients as candidates for safe and effective treatment of symptoms of epilepsy or epileptic encephalopathy with fenfluramine or a pharmaceutically acceptable salt thereof.

### SUMMARY

According to a first aspect of the present invention, provided herein is a method of identifying a patient population as candidates for effective treatment of symptoms of epilepsy or epileptic encephalopathy with fenfluramine or a pharmaceutically acceptable salt thereof, by selecting patients diagnosed with epilepsy or epileptic encephalopathy; assessing the patients for symptoms of pulmonary hypertension; treating patients at risk of or exhibiting symptoms of pulmonary hypertension; re-assessing the patients for symptoms of pulmonary hypertension; and identifying treated and re-assessed patients not exhibiting pulmonary hypertension as candidates for effective treatment of epilepsy or epileptic encephalopathy with fenfluramine or a pharmaceutically acceptable salt thereof.

Another aspect of the invention includes a kit, including a fenfluramine formulation, a package, a package insert comprising content warning against administration to an epileptic patient exhibiting symptoms of pulmonary hypertension.

Another aspect of the invention includes a kit, including a container comprising a plurality of doses of a formulation comprising a pharmaceutically acceptable carrier and an active ingredient comprising fenfluramine; and instructions for treating the patient diagnosed with epilepsy or epileptic encephalopathy, in which the instructions warn against administering the formulation to the patient if the patient is exhibiting signs of pulmonary hypertension.

These and other objects, advantages, and features of the invention will become apparent to those persons skilled in the art upon reading the details of the methods of identifying a patient population as candidates for treatment of symptoms of epilepsy or epileptic encephalopathy with fenfluramine, as more fully described below.

### DETAILED DESCRIPTION

Before the present method, kits and formulations are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a seizure” includes a plurality of such seizures and reference to “the formulation” includes reference to one or more formulations and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present disclosure relates to a method of identifying a patient population amenable to the treatment of symptoms of epilepsy or epileptic encephalopathy with fenfluramine. More specifically, the disclosure relates to methods of identifying a population of patients as candidates for effective treatment of symptoms of epilepsy or epileptic encephalopathy with fenfluramine, by pre-selecting epileptic patients and assessing them for being at risk of or exhibiting symptoms of PH; treating patients at risk or exhibiting symptoms of PH with one or more agents to prevent or treat the PH, prior to administration of fenfluramine to treat the symptoms of epilepsy; re-assessing the selected and treated patients for symptoms of PH; and identifying treated and re-assessed patients not exhibiting symptoms of PH as candidates for effective treatment of epilepsy or epileptic encephalopathy with fenfluramine or a pharmaceutically acceptable salt thereof.

Fenfluramine, i.e. 3-trifluoromethyl-N-ethylamphetamine is an amphetamine derivative having the structure:

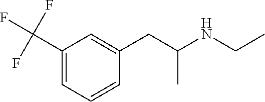 [SMILES: CCNC(C)Cc1cccc(C(F)(F)F)c1]

### Systematic (IUPAC) Name

### (RS)—N-ethyl-1-[3-(trifluoromethyl)phenyl]propan-2-amine

Fenfluramine was first marketed in the US in 1973 to treat obesity. However, in 1997, it was withdrawn from the US and global market as its use was associated with the onset of cardiac valvulopathy and pulmonary hypertension (PH). Subsequently, the drug was withdrawn from sale globally and is no longer indicated for use in any therapeutic area. Without being bound by theory, the adverse effects associated with the use of fenfluramine as an anorexic agent are thought to be attributable to the interaction of fenfluramine's major metabolite norfenfluramine with the 5-HT2B receptor, which is associated with heart valve hypertrophy.

Fenfluramine is metabolized in vivo into norfenfluramine by cytochrome P450 enzymes in the liver. Cytochrome P450 enzymes such as CYP2D6, CYP2B6 and CYP1A2 are primarily responsible for the production of norfenfluramine from fenfluramine in humans. The enzymes CYP2C9, CYP2C19 and CYP3A4 are also involved. Such metabolism includes cleavage of an N-ethyl group to produce norfenfluramine as shown below.

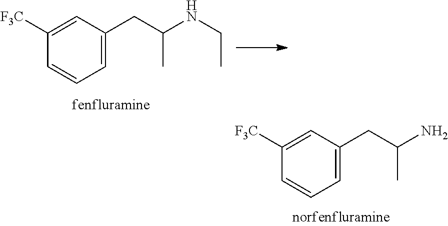 [SMILES: CCNC(C)Cc1cccc(C(F)(F)F)c1 ; CC(N)Cc1cccc(C(F)(F)F)c1]

Despite past cardiovascular safety concerns that arose when high doses of fenfluramine were used for treatment of adult obesity, attempts have been made to identify further therapeutic uses for that product, while weighing the known cardiovascular risks of fenfluramine against potential therapeutic benefits. Disorders for which new treatment options are sorely needed is epilepsy or epileptic encephalopathy, and in particular, epilepsy syndromes which are refractory to known treatments. Epilepsy is a condition of the brain marked by a susceptibility to recurrent seizures. There are numerous causes of epilepsy including, but not limited to birth trauma, perinatal infection, anoxia, infectious diseases, ingestion of toxins, tumors of the brain, inherited disorders or degenerative disease, head injury or trauma, metabolic disorders, cerebrovascular accident and alcohol withdrawal.

Prior to the inventor's work, investigation of fenfluramine's efficacy in epilepsy patients, while showing some initial promise, was far from definitive, and shared a common paradigm, i.e., that fenfluramine's primary effects were on behaviors that caused or induced seizures, not treating or preventing the seizure itself.

For example, Aicardi and Gastaut (New England Journal of Medicine (1985), 313:1419 and Archives of Neurology (1988) 45:923-925) reported four cases of self-induced photosensitive seizures, i.e., seizures caused by patients purposely staring into bright lights or the sun, that responded to treatment with fenfluramine.

Clemens, in Epilepsy Research (1988) 2:340-343 reported a case study wherein a boy suffering pattern sensitivity-induced seizures that were resistant to anticonvulsive treatment was treated with fenfluramine to curb the patient's compulsive seizure-inducing behavior. Fenfluramine reportedly successfully terminated these self-induced seizures and the author concluded that this was because fenfluramine blocked the seizure-sensitive triggering mechanism, i.e., not by treating the seizure itself.

In Neuropaediatrics, (1996); 27(4):171-173, Boel and Casaer reported on a study on the effects of fenfluramine on children with refractory epilepsy, all of whom exhibited compulsive seizure-inducing behavior. They observed that when fenfluramine was administered at a dose of 0.5 to 1 mg/kg/day, this resulted in a reduction in the number of seizures experienced by the patients, and concluded that “this drug could have significant anti-epileptic activity in a selected group of young patients with idiopathy or symptomatic generalized epilepsy, namely, children with self-induced seizures.” The authors noted that “[i]t may well be that fenfluramine has no direct antiepileptic activity but acts through its effect on the compulsion to induce seizures.” Hence the authors seemed to suggest that fenfluramine affected behavior and not the seizure itself.

In a letter to Epilepsia, published in that journal (Epilepsia, 43(2):205-206, 2002), Boel and Casaer commented that fenfluramine appeared to be of therapeutic benefit in patients with intractable epilepsy and self-induced seizures. However, the authors did not attribute fenfluramine's efficacy to generalized anti-seizure activity.

A large number of subtypes of epilepsy have been characterized, each with its own unique clinical symptoms, signs, and phenotype, underlying pathophysiology and distinct responses to different treatments. The most recent version, and the one that is widely accepted in the art, is that adopted by the International League Against Epilepsy's (“ILAE”) Commission on Classification and Terminology [See e.g., Berg et al., “Revised terminology and concepts for organization of seizures,” Epilepsia, 51(4):676-685 (2010)]:
I. Electroclinical syndromes arranged by age at onset:
   A. Neonatal period (1. Benign familial neonatal epilepsy (BFNE), 2. Early myoclonic encephalopathy (EME), 3. Ohtahara syndrome),
   B. Infancy (1. Epilepsy of infancy with migrating focal seizures, 2. West syndrome, 3. Myoclonic epilepsy in infancy (MEI), 4. Benign infantile epilepsy, 5. Benign familial infantile epilepsy, 6. Dravet syndrome, 7. Myoclonic encephalopathy in nonprogressive disorders),
   C. Childhood (1. Febrile seizures plus (FS+) (can start in infancy), 2. Panayiotopoulos syndrome, 3. Epilepsy with myoclonic atonic (previously astatic) seizures, 4. Benign epilepsy with centrotemporal spikes (BECTS), 5. Autosomal-dominant nocturnal frontal lobe epilepsy (ADNFLE), 6. Late onset childhood occipital epilepsy (Gastaut type), 7. Epilepsy with myoclonic absences, 8. Lennox-Gastaut syndrome, 9. Epileptic encephalopathy with continuous spike-and-wave during sleep (CSWS), 10. Landau-Kleffner syndrome (LKS), 11. Childhood absence epilepsy (CAE));
   D. Adolescence-Adult (1. Juvenile absence epilepsy (JAE), 2. Juvenile myoclonic epilepsy (JME), 3 Epilepsy with generalized tonic-clonic seizures alone, 4. Progressive myoclonus epilepsies (PME), 5. Autosomal dominant epilepsy with auditory features (ADEAF), 6. Other familial temporal lobe epilepsies, E. Less specific age relationship (1. Familial focal epilepsy with variable foci (childhood to adult), 2. Reflex epilepsies);
II. Distinctive constellations:
   A. Mesial temporal lobe epilepsy with hippocampal sclerosis (MTLE with HS),
   B. Rasmussen syndrome,
   C. Gelastic seizures with hypothalamic hamartoma,
   D. Hemiconvulsion-hemiplegia-epilepsy,
   E. Other epilepsies, distinguished by 1. presumed cause (presence or absence of a known structural or metabolic condition, then 2. primary mode of seizure onset (generalized vs. focal);
III. Epilepsies attributed to and organized by structural-metabolic causes:
   A. Malformations of cortical development (hemimegalencephaly, heterotopias, etc.),
   B. Neurocutaneous syndromes (tuberous sclerosis complex, Sturge-Weber, etc.),
   C. Tumor,
   D. Infection,
   E. Trauma;
IV. Angioma: A. Perinatal insults, B. Stroke, C. Other causes;
V. Epilepsies of unknown cause;
VI. Conditions with epileptic seizures that are traditionally not diagnosed as a form of epilepsy per se; A. Benign neonatal seizures (BNS); and B. Febrile seizures (FS).

See Berg et. al, “Revised terminology and concepts for organization of seizures,” Epilepsia, 51(4):676-685 (2010)).

Part V of the ILAE classification scheme underscores the fact that the list is far from complete, and that there are still subtypes of epilepsy or epileptic encephalopathy that have not yet been fully characterized, or that remain unrecognized as distinct syndromes.

Without being bound by theory, fenfluramine has been known to trigger the release of serotonin (5-HT) in the brain due to disruption of its vesicular storage and to inhibit serotonin reuptake. However, until recently, it was not known that fenfluramine's mechanism of action made it suitable for the treatment of epilepsy or epileptic encephalopathy (e.g., Dravet syndrome or Lennox-Gastaut syndrome (LGS)). In fact, there are no scientific publications demonstrating or even hypothesizing that 5-HT abnormalities are a possible underlying pathophysiologic cause for LGS or are causally related to the associated seizures in this specific epilepsy condition. Furthermore, since there has been no scientific hypothesis relating serotonin abnormalities in LGS, there are no studies nor even individual case reports in the medical literature which describe attempts to treat LGS using medications that interacts with serotonin. The lack of data or even speculation in the literature regarding the use of fenfluramine or serotonergic agents in general to treat LGS are facts that strongly support the unexpected nature of this discovery: given that LGS is a devastating refractory epilepsy condition and the number of people affected, investigators would be strongly motivated to investigate any treatment they perceived as having any potential for efficacy.

Lennox-Gastaut syndrome (LGS) was first described in 1960, and named for neurologists William G. Lennox (Boston, USA) and Henri Gastaut (Marseille, France). It is a difficult-to-treat form of childhood-onset epilepsy that most often appears between the second and sixth year of life, although it can occur at an earlier or later age. LGS is characterized by frequent seizures and different seizure types; it is typically accompanied by developmental delay and psychological and behavioral problems. In children, common causes of LGS include perinatal brain injury, brain malformations such as tuberous sclerosis or cortical dysplasia, central nervous system (CNS) infection, and degenerative or metabolic disorders of the nervous system.

Daily multiple seizures of different types are typical in LGS. Also typical is the broad range of seizures that can occur. The most common seizure types are tonic-axial, atonic, and absence seizures, but myoclonic, generalized tonic-clonic, and focal seizures can also occur in any LGS patient. Atonic, atypical absence, tonic, focal, and tonic-clonic seizures are also common. Additionally, many LGS patients will have status epilepticus, often of the nonconvulsive type, which is characterized by dizziness, apathy, and unresponsiveness. Further, most patients have atonic seizures, also called drop seizures, which cause their muscles to go limp and result in the patient suddenly and unexpectedly to fall to the ground, often causing significant injury, which is why patients often wear a helmet to prevent head injury.

In addition to daily multiple seizures of various types, children with LGS frequently have arrested/slowed psycho-motor development and behavior disorders.

The syndrome is also characterized by a specific finding on electroencephalogram (EEG), specifically an interictal (i.e., between-seizures) slow spike-wave complexes and fast activity during sleep.

### Diagnosis

LGS is a syndrome and hence its diagnosis is based on the presence of specific clinical symptoms, signs, and laboratory tests. LGS is typically identified by a triad of features including multiple types of seizures, mental retardation or regression and abnormal EEG with generalized slow spike and wave discharges. Physicians use EEG to assist in diagnosing LGS. Diagnosis may be difficult at the onset of the initial symptom(s) because the triad of features associated with LGS, such as tonic seizures, may not be fully established, and EEG during sleep is required to confirm the condition. Thus, even though there may be overlap in clinical presentation with other epilepsies, LGS is agreed to be a well-defined distinct diagnosis by both the International League Against Epilepsy (ILAE), considered the world's leading expert medical society on epilepsy, and the FDA.

The diagnosis of LGS is more obvious when the patient suffers frequent and manifold seizures, with the classic pattern on the electro-encephalogram (EEG), i.e., a slowed rhythm with Spike-wave-pattern, or with multifocal and generalizing sharp-slow-wave-discharges at 1.5-2.5 Hz. During sleep, tonic patterns (fast activity) can often be seen.

General medical investigation usually reveals developmental delay and cognitive deficiencies in children with LGS. These may precede development of seizures, or require up to two years after the seizures begin, in order to become apparent.

There may be multiple etiologies for LGS, including genetic, structural, metabolic or unknown. Approximately one-quarter have no prior history of epilepsy, neurological abnormality or developmental delay prior to the onset of LGS symptoms. Underlying pathologies causing LGS may include encephalitis and/or meningitis, brain malformations (e.g., cortical dysplasias), birth injury, hypoxia-ischemia injury, frontal lobe lesions, and trauma.

An important differential diagnosis is ‘Pseudo-Lennox-Syndrome’, also called atypical benign partial epilepsy of childhood, which differs from LGS, in that there are no tonic seizures; sleeping EEG provides the best basis for distinguishing between the two. In addition, ‘Pseudo-Lennox-Syndrome’ has an entirely different etiology and prognosis than LGS.

The use of several common first-line treatments is based on clinical experience or conventional wisdom; examples include broad spectrum anti-convulsant medications, such as valproic acid, and benzodiazepines, most often clonazepam and clobazam. A few drugs have been proven effective for some patients for certain seizure types by double-blind placebo-controlled studies; examples include clobazam, lamotrigine, topiramate, felbamate, and rufinamide, although most patients continue to have significant seizures even while taking these medications. Second-line medications currently in use, such as zonisamide, are prescribed based on results of some open-label uncontrolled studies. The ketogenic diet may be useful in some patients with LGS refractory to medical treatment. Surgical options for LGS include corpus callostomy (for drop attacks), vagus nerve stimulation, and focal cortical resection (in the presence of a single resectable lesion). However, it should be noted that significant improvement from any of these therapies alone or in combination is a rare occurrence.

Despite the severity of LGS's symptoms and the frequency with which it occurs (it accounts for up to 10% of all childhood epilepsies), there is currently no standard evidence-based treatment for the disease. A comprehensive review of the literature [see Hancock E C & Cross J H, Treatment of Lennox-Gastaut syndrome (Review), published in The Cochrane Library 2013, Issue 2] discovered only nine randomized controlled trials which evaluated the pharmaceutical treatment of the syndrome. The authors concluded that there is a paucity of research and “ . . . that no monotherapy (to date) has been shown to be highly effective in this syndrome.” Id at page 12. The authors further concluded that “[t]he optimum treatment for LGS remains uncertain and no study to date has shown any one drug to be highly efficacious”. Id at page 12.

### Treatment

Many different treatments are currently used in the treatment of this disorder and many more have been tried in the past, most often with little success. A variety of therapeutic approaches are currently used in LGS, including conventional antiepileptic medications, diet and surgery, however the evidence supporting these therapies is not robust and treatment remains most often ineffective.

After extensive research, it was unexpectedly found that fenfluramine can be used effectively to treat, or at least minimize the symptoms of epilepsy or epileptic encephalopathy.

However, as described above, it has been suggested that appetite suppressants such as dexfenfluramine may precipitate or hasten the development of PH, and thus, using fenfluramine to treat symptoms of epilepsy of epileptic encephalopathy may be problematic and contraindicated in subjects having a high risk of, or currently experiencing pulmonary hypertension.

For example, in a small study of 73 patients with PH, fifteen (20%) had used fenfluramine, and all were women. In 10 of those 15 women (67%), there was a close temporal relation between fenfluramine use and the development of exertional dyspnoea. In this study, pulmonary hypertension was defined as a mean resting pulmonary artery pressure >25 mm Hg during right heart catheterization, with a mean pulmonary wedge pressure below 12 mm Hg. Initial right heart catheterization in the 15 women showed severe resting pulmonary hypertension (mean (SD)) with pulmonary artery pressure (PAP) 57 (9) mm Hg, cardiac index 2·1 (0·5) 1/min/m², and pulmonary vascular resistance (PVR) 29 (10) U/m². Short-term epoprostenol infusion produced a significant vasodilator response in 10 patients (mean fall in PVR 24 (15%) compared with control values). Three fenfluramine users with PH showed spontaneous clinical and hemodynamic improvement 3, 6 and 12 months after drug withdrawal but there was no significant difference in overall survival (transplant recipients excluded) between fenfluramine users and controls. Histological examination of lung tissue from five women who had used fenfluramine and 22 controls with PH showed features typical of advanced plexogenic pulmonary arteriopathy in all. Overall, it was observed that these results did not accord with earlier reports that PH associated with fenfluramine is less severe and has a better outcome. Fenfluramine was suggested to be an etiological agent that can precipitate or hasten the development of PH. (Brenot, et al., (1993) Br. Heart J. 70(6):537-541).

It has been suggested that, at least in some cases, pulmonary hypertension may arise more from pulmonary vascular remodeling rather than vasoconstriction. Pathologic studies have observed vascular remodeling and a limited decrease in pulmonary vascular resistance during treatment of IPAH. However, in a small subset (5-10%) of patients, a dramatic hemodynamic improvement has also been observed in response to acute exposure to pulmonary vasodilators; approximately one half of these “vasodilator-responsive” patients improved clinically with calcium-channel blocker therapy. The disparity in outcomes has fueled speculation that vasodilator-responsive and nonresponsive IPAH may be distinct diseases. (Brittain and Hemnes, Ann. Intern. Med. 2015; 162(2):148-149).

To avoid doubt, the term “prevention” of a symptom of epilepsy or epileptic encephalopathy (e.g., seizures) means the total or partial prevention (inhibition) of the symptom (seizures). Ideally, the methods of the present invention result in a total prevention of seizures. However, the invention also encompasses methods in which the instances of seizures are decreased in frequency by at least 40%, at least 50%, at least 55%, at least 60%, at least 65%, at least 70%, at least 75%, at least 80%, at least 85%, or at least 90%. In addition, the invention also encompasses methods in which the instances of seizures are decreased in duration or severity by at least 40%, at least 50%, at least 55%, at least 60%, at least 65%, at least 70%, at least 75%, at least 80%, at least 85%, or at least 90%.

The present disclosure relates to methods of identifying a population of patients amenable to the treatment of symptoms of epilepsy or epileptic encephalopathy with fenfluramine. More specifically, the disclosure relates to methods of identifying a patient population as candidates for effective treatment of symptoms of epilepsy or epileptic encephalopathy with fenfluramine, by pre-selecting epileptic patients and assessing them for being at risk of or exhibiting symptoms of pulmonary hypertension (PH); treating patients at risk or exhibiting symptoms of PH with one or more agents to prevent or treat the PH, prior to administration of fenfluramine to treat the symptoms of epilepsy; re-assessing the selected and treated patients for symptoms of PH; and identifying treated and re-assessed patients not exhibiting symptoms of PH as candidates for effective treatment of epilepsy or epileptic encephalopathy with fenfluramine or a pharmaceutically acceptable salt thereof. In certain cases it relates to determining which patients should be excluded from treatment, such as patients who do not respond to administration of agents to treat PH or to patients who have pulmonary artery pressures at or above certain thresholds.

Thus, in some aspects, provided herein is a method of identifying a population of patients as candidates for effective treatment of symptoms of epilepsy or epileptic encephalopathy with fenfluramine or a pharmaceutically acceptable salt thereof, by selecting patients diagnosed with epilepsy or epileptic encephalopathy; assessing the patients for risk or exhibiting symptoms of pulmonary hypertension; treating patients at risk or exhibiting symptoms of pulmonary hypertension; re-assessing the patients for symptoms of pulmonary hypertension; and identifying treated and re-assessed patients not exhibiting pulmonary hypertension as candidates for effective treatment of epilepsy or epileptic encephalopathy with fenfluramine or a pharmaceutically acceptable salt thereof.

In some embodiments, the assessing includes determining whether the patients have one or more risk factors of pulmonary hypertension selected from a family history of PH, genetic predisposition, primary pulmonary hypertension, heart failure, left ventricular failure, valvular heart disease, history of blood clots in the lungs/pulmonary embolism, HIV/AIDS, sickle cell disease, cocaine use, use of appetite suppressants, COPD, sleep apnea, living at high altitudes, and problems with the mitral valve. Early/initial symptoms of PH include exertional dyspnea, lethargy, fatigue (alone or in combination with other symptoms and/or risk factors). Late symptoms include exertional chest pain, angina, exertional syncope, peripheral edema, anorexia, abdominal pain (alone or in combination with other symptoms and/or risk factors).

In some embodiments, the assessing includes genetically testing the selected patients diagnosed with epilepsy or epileptic encephalopathy and identifying genetic risk factors for pulmonary hypertension.

In some embodiments the assessing includes measuring pulmonary artery systolic pressure or mean pulmonary artery pressure in a patient.

In some embodiments, the treating of patients occurs when they are assessed to have a pulmonary artery systolic pressure ≥28 mm of Hg or mean pulmonary artery pressure of ≥20 mm of Hg. as estimated based on echocardiography, or actual pressure measurements by right heart catheterization (via a Swan-Ganz catheter inserted through the right side of the heart). Pulmonary hypertension is defined as a mean pulmonary arterial pressure (PAP) of at least 25 mm Hg at rest, and PAH is defined as precapillary pulmonary hypertension (i.e. mean PAP ≥25 mm Hg with pulmonary arterial occlusion pressure [PAOP]≤15 mm Hg and pulmonary vascular resistance [PVR]≥3 Wood Units. (see Hoeper et al.)

In some embodiments, the treating of patients for symptoms of pulmonary hypertension includes administering diuretics (e.g., furosemide, spironolactone, amiloride) and/or vasodilators (e.g., epoprostenol, tadalafil, ambrisentan, sildenafil, iloprost, alprostadil, treprostinil).

In some embodiments, the patients assessed and identified as having genetic risk factors for pulmonary hypertension are treated prophylactically with one or more of furosemide, spironolactone, amiloride, epoprostenol, tadalafil, ambrisentan, sildenafil, iloprost, alprostadil or treprostinil to mitigate the risk.

In some embodiments, the treating of patients at risk or exhibiting symptoms of pulmonary hypertension includes reducing sodium intake.

In some embodiments, the treating of patients at risk or exhibiting symptoms of pulmonary hypertension includes a regimen of diet and increased exercise.

In some embodiments, the patient diagnosed with epilepsy has Dravet syndrome or Lennox-Gastaut syndrome.

In some embodiments, the patient diagnosed with epilepsy has epileptic encephalopathy.

In some embodiments, a symptom of the epileptic encephalopathy is seizure, and wherein the fenfluramine is formulated with a pharmaceutically acceptable carrier and an effective dose is less than 10.0 mg/kg/day to 0.01 mg/kg/day.

In some embodiments, the patients diagnosed with epilepsy or epileptic encephalopathy are adults (18 years of age or older).

Another aspect of the present disclosure includes a kit, comprising a fenfluramine formulation, a package, and a package insert comprising content warning against administration to an epileptic patient exhibiting symptoms of pulmonary hypertension.

Another aspect of the present disclosure includes a kit, comprising a container including a plurality of doses of a formulation and a pharmaceutically acceptable carrier, and an active ingredient comprising fenfluramine; and instructions for treating the patient diagnosed with epilepsy or epileptic encephalopathy, wherein the instructions warn against administering the formulation to the patient if the patient is exhibiting signs of pulmonary hypertension.

In some embodiments, a patient having risk factors for developing PH is treated for and prevented from developing (i.e., does not develop) PH, and is subsequently treated with fenfluramine in an amount effective to treat the symptoms of epilepsy or epileptic encephalopathy. In some embodiments, a patient exhibiting symptoms of PH is treated and the PH symptoms are reduced, ameliorated or eliminated, and the patient is subsequently treated with fenfluramine in an amount effective to treat the symptoms of epilepsy or epileptic encephalopathy. In some embodiments, a patient exhibiting symptoms of PH is treated and the PH symptoms are not reduced, ameliorated or eliminated, and the patient then is excluded from treatment with fenfluramine, as contraindicated.

The method may be carried out in a process wherein the patient is first subjected to a series of tests to confirm diagnosis of epilepsy or epileptic encephalopathy (e.g.) LGS.

Fenfluramine can be administered in the form of the free base, or in the form of a pharmaceutically acceptable salt, for example selected from the group consisting of hydrochloride, hydrobromide, hydroiodide, maleate, sulphate, tartrate, acetate, citrate, tosylate, succinate, mesylate and besylate. Further illustrative pharmaceutically acceptable salts can be found in Berge et al., J. Pharm. Sci. (1977) 68(1): 1-19.

Fenfluramine for use in the methods of the present invention may be produced according to any pharmaceutically acceptable process known to those skilled in the art. Examples of processes for synthesizing fenfluramine are provided in the following documents: GB1413070, GB1413078 and EP441160.

The dose of fenfluramine to be used in a method of the present invention can be provided in the form of a kit, including instructions for using the dose in one or more of the methods of the present invention. In certain embodiments, the kit can additionally comprise a dosage form comprising one or more co-therapeutic agents.

The fenfluramine can be formulated as an oral liquid or a solid oral dosage form or a transdermal patch.

The method may be carried out as a co-treatment with a different pharmaceutically active compound. The treatment may be carried out to relieve symptoms of epilepsy or epileptic encephalopathy by the co-administration of fenfluramine and a co-therapeutic agent. For example, the fenfluramine may be co-administered with an effective dose of one or more other pharmaceutical drugs such as a co-therapeutic agent selected from the group consisting of cannabidiol, carbamazepine, ethosuximide, fosphenytoin, lamotrigine, levetiracetam, phenobarbital, progabide, topiramate, stiripentol, valproic acid, valproate, verapamil, and benzodiazepines such as clobazam, clonazepam, diazepam, ethyl loflazepate, lorazepam, midazolam. Use of a pharmaceutically acceptable salt or base of a co-therapeutic agent is also contemplated.

As a specific example of a combination of co-therapeutic agents with fenfluramine, the co-therapeutic agent may be any one of or all three of stiripentol, clobazam, and valproate. The fenfluramine may be administered in the amount of 0.8 mg/kg of patient body weight and co-administered with 3500 mg of stiripentol, 20 mg of clobazam, and 25 mg per kg of valproate. Each of those amounts may be increased to twice, three times, five times, or ten times that amount or decreased by 10%, 50%, or 75%.

The co-therapeutic agents have recommended dosing amounts. Those recommended dosing amounts are provided within the most current version of the Physician's Desk Reference (PDR) or online at emedicine.medscape.com, both of which are incorporated herein by reference specifically with respect to the co-therapeutic agents listed above and more specifically with respect to the dosing amounts recommended for those drugs.

In connection with the present invention, the co-therapeutic agent can be used in the recommended dosing amount or can be used in a range of from 1000 to 100 times, 1/10 to 10 times, ⅕ to 5 times, or ½ to twice the recommended dosing amount or any incremental 1/10 amount in between those ranges.

As specific examples of forms of epilepsy or epileptic encephalopathy to be treated in the methods disclosed herein are Dravet syndrome and Lennox-Gastaut syndrome (LGS). Also described herein is a use of a fenfluramine formulation for treating and or preventing symptoms of Dravet syndrome or LGS in a patient, which use may include placing the fenfluramine in a liquid solution and withdrawing that liquid solution into a graduated syringe.

An example of an effective dose of fenfluramine or pharmaceutically acceptable salt to be administered to the patient, the dose is administered in an amount in the range of from 10.0 mg/kg/day to about 0.01 mg/kg/day, or administered at 120 mg or less; or 60 mg or less; or 30 mg or less; or 20 mg or less, and may be administered in the presence or in the absence of the administration of any other pharmaceutically active compound.

The method may be carried out wherein the effective dose is administered in a form selected from the group consisting of oral, injectable, transdermal, buccal, inhaled, nasal, rectal, vaginal, or parental, and wherein the formulation is oral, the formulation may be liquid which may be a solution or a suspension may be present within a container closed with a cap connected to a syringe graduated to determine the volume extracted from the container wherein the volume extracted relates to the amount of fenfluramine in a given liquid volume of formulation e.g. one milliliter of formulation contains 2.5 mg of fenfluramine. In another aspect of the invention, the method is administered in a solid oral formulation in the form of a tablet, capsule, lozenge, or sachet.

The kit may be a kit for treating Dravet syndrome in a patient diagnosed with Dravet syndrome wherein the kit comprises a formulation comprising a pharmaceutically acceptable carrier and an active ingredient comprising fenfluramine and instructions for treating a patient diagnosed with Dravet syndrome by administering the formulation to the patient. The kit may be a kit for treating Lennox-Gastaut syndrome (LGS) in a patient diagnosed with LGS wherein the kit comprises a formulation comprising a pharmaceutically acceptable carrier and an active ingredient comprising fenfluramine and instructions for treating a patient diagnosed with LGS by administering the formulation to the patient.

The kit may consist of an oral liquid formulation in a container and a syringe with instructions, wherein the concentration of the fenfluramine in the liquid is calibrated based on calibrations on the syringe and includes calibrations wherein a milliliter of solution equates to a known amount of fenfluramine such as 0.1 mg, 0.2 mg etc., to 1.0 mg.

In some embodiments, the kit includes instructions relating to dosing the patient based on patient weight and volume of solution based on the concentration of fenfluramine in the solution.

In embodiments of the disclosure, any effective dose of fenfluramine can be employed. However, surprisingly low doses of fenfluramine have been found by the inventors to be effective, particularly for inhibiting or eliminating seizures in epilepsy or epileptic encephalopathy patients.

DOSE BY WEIGHT (MG/KG/DAY): Thus in some cases, in a preferred embodiment of the invention, a daily dose of less than about 10 mg/kg/day, such as less than about 10 mg/kg/day, less than about 9 mg/kg/day, less than about 8 mg/kg/day, less than about 7 mg/kg/day, less than about 6 mg/kg/day, less than about 5 mg/kg/day, less than about 4 mg/kg/day, less than about 3.0 mg/kg/day, less than about 2.5 mg/kg/day, less than about 2.0 mg/kg/day, less than about 1.5 mg/kg/day, less than about 1.0 mg/kg/day, such as about 1.0 mg/kg/day, about 0.95 mg/kg/day, about 0.9 meg/kg/day, about 0.85 mg/kg/day, about 0.85 mg/kg/day, about 0.8 mg/kg/day, about 0.75 mg/kg/day, about 0.7 mg/kg/day, about 0.65 mg/kg/day, about 0.6 mg/kg/day, about 0.55 mg/kg/day, about 0.5 mg/kg/day, about 0.45 mg/kg/day, about 0.4 mg/kg/day, about 0.350 mg/kg/day, about 0.3 mg/kg/day, about 0.25 mg/kg/day, about 0.2 mg/kg/day, about 0.15 mg/kg/day to about 0.1 mg/kg/day, about 0.075 mg/kg/day, about 0.05 mg/kg/day, about 0.025 mg/kg/day, about 0.0225 mg/kg/day, about 0.02 mg/kg/day, about 0.0175 mg/kg/day, about 0.015 mg/kg/day, about 0.0125 mg/kg/day, or about 0.01 mg/kg/day is employed.

Put differently, a preferred dose is less than about 10 to about 0.01 mg/kg/day. In some cases the dose is less than about 10.0 mg/kg/day to about 0.01 mg/kg/day, such as less than about 5.0 mg/kg/day to about 0.01 mg/kg/day, less than about 4.5 mg/kg/day to about 0.01 mg/kg/day, less than about 4.0 mg/kg/day to about 0.01 mg/kg/day, less than about 3.5 mg/kg/day to about 0.01 mg/kg/day, less than about 3.0 mg/kg/day to about 0.01 mg/kg/day, less than about 2.5 mg/kg/day to about 0.01 mg/kg/day, less than about 2.0 mg/kg/day to about 0.01 mg/kg/day, less than about 1.5 mg/kg/day to about 0.01 mg/kg/day, or less than about 1.0 mg/kg/day to 0.01 mg/kg/day, such as less than about 0.9 mg/kg/day, less than about 0.8 mg/kg/day, less than about less than about 0.7 mg/kg/day, less than about 0.6 mg/kg/day to about 0.01 mg/kg/day, less than about 0.5 mg/kg/day to about 0.01 mg/kg/day, less than about 0.4 mg/kg/day to about 0.01 mg/kg/day, less than about 0.3 mg/kg/day to about 0.01 mg/kg/day, or less than about 0.2 mg/kg/day to about 0.01 mg/kg/day.

As indicated above the dosing can be based on the weight of the patient. However, for convenience the dosing amounts may be preset such as in the amount of 1.0 mg, 2.5 mg, 5 mg, 10 mg, 15 mg, 20 mg, 30 mg, 40 mg, or 50 mg. In certain instances, the dosing amount may be preset such as in the amount of about 0.25 mg to about 5 mg, such as about 0.25 mg, about 0.5 mg, about 0.75 mg, about 1.0 mg, about 1.25 mg, about 1.5 mg, about 1.75 mg, about 2.0 mg, about 2.25 mg, about 2.5 mg, about 2.75 mg, about 3.0 mg, about 3.25 mg, about 3.5 mg, about 3.75 mg, about 4.0 mg, about 4.25 mg, about 4.5 mg, about 4.75 mg, or about 5.0 mg.

The dosing amounts described herein may be administered one or more times daily to provide for a daily dosing amount, such as once daily, twice daily, three times daily, or four or more times daily, etc.

In certain embodiments, the dosing amount is a daily dose of 30 mg or less, such as 30 mg, about 29 mg, about 28 mg, about 27 mg, about 26 mg, about 25 mg, about 24 mg, about 23 mg, about 22 mg, about 21 mg, about 20 mg, about 19 mg, about 18 mg, about 17 mg, about 16 mg, about 15 mg, about 14 mg, about 13 mg, about 12 mg, about 11 mg, about 10 mg, about 9 mg, about 8 mg, about 7 mg, about 6 mg, about 5 mg, about 4 mg, about 3 mg, about 2 mg, or about 1 mg. In general the smallest dose which is effective should be used for the particular patient. In some cases, the dose is generally well below the dosing used in weight loss.

Methods of administration may include administration via enteral routes, such as oral, buccal, sublingual, and rectal; topical administration, such as transdermal and intradermal; and parenteral administration. Suitable parenteral routes include injection via a hypodermic needle or catheter, for example, intravenous, intramuscular, subcutaneous, intradermal, intraperitoneal, intraarterial, intraventricular, intrathecal, and intracameral injection and non-injection routes, such as intravaginal rectal, or nasal administration. In certain embodiments, it may be desirable to administer one or more compounds of the invention locally to the area in need of treatment. This may be achieved, for example, by local infusion during, topical application, by injection, by means of a catheter, by means of a suppository, or by means of an implant, said implant being of a porous, non-porous, or gelatinous material, including membranes, such as sialastic membranes, or fibers.

DOSAGE FORMS/ROUTE OF ADMINISTRATION: The dose of fenfluramine administered according to the methods of the present invention can be administered systemically or locally. The dose of fenfluramine administered according to the methods of the present invention can be formulated in any pharmaceutically acceptable dosage form including, but not limited to (a) oral dosage forms such as tablets including orally disintegrating tablets, capsules, and lozenges, oral solutions or syrups, oral emulsions, oral gels, oral films, buccal liquids, powder e.g. for suspension, and the like; (b) injectable dosage forms; (c) transdermal dosage forms such as transdermal patches, ointments, creams; (c) inhaled dosage forms; and/or (e) nasally, (f) rectally, (g) vaginally administered dosage forms.

DOSGE FORM/FREQUENCY OF ADMINISTRATION: Such dosage forms can be formulated for once a day administration, or for multiple daily administrations (e.g. 2, 3 or 4 times a day administration). Alternatively, for convenience, dosage forms can be formulated for less frequent administration (e.g., monthly, bi-weekly, weekly, every fourth day, every third day, or every second day), and formulations which facilitate extended release are known in the art.

DOSAGE FORMS/PREPARATION, COMPONENTS: The dosage form of fenfluramine employed in the methods of the present invention can be prepared by combining fenfluramine or a pharmaceutically acceptable salt thereof with one or more pharmaceutically acceptable diluents, carriers, adjuvants, and the like in a manner known to those skilled in the art of pharmaceutical formulation.

ORAL DOSAGE FORMS/SUITABLE FORMULATION TYPES & COMPONENTS THEREOF: In some embodiments, formulations suitable for oral administration can include (a) liquid solutions, such as an effective amount of the compound dissolved in diluents, such as water, or saline; (b) capsules, sachets or tablets, each containing a predetermined amount of the active ingredient (fenfluramine), as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

ORAL DOSAGE FORMS/EXCIPIENTS: For an oral solid pharmaceutical formulation, suitable excipients include pharmaceutical grades of carriers such as mannitol, lactose, glucose, sucrose, starch, cellulose, gelatin, magnesium stearate, sodium saccharine, and/or magnesium carbonate. For use in oral liquid formulations, the composition may be prepared as a solution, suspension, emulsion, or syrup, being supplied either in solid or liquid form suitable for hydration in an aqueous carrier, such as, for example, aqueous saline, aqueous dextrose, glycerol, or ethanol, preferably water or normal saline. If desired, the composition may also contain minor amounts of non-toxic auxiliary substances such as wetting agents, emulsifying agents, or buffers.

By way of illustration, the fenfluramine composition can be admixed with conventional pharmaceutically acceptable carriers and excipients (i.e., vehicles) and used in the form of aqueous solutions, tablets, capsules, elixirs, suspensions, syrups, wafers, and the like. Such pharmaceutical compositions contain, in certain embodiments, from about 0.1% to about 90% by weight of the active compound, and more generally from about 1% to about 30% by weight of the active compound. The pharmaceutical compositions may contain common carriers and excipients, such as corn starch or gelatin, lactose, dextrose, sucrose, microcrystalline cellulose, kaolin, mannitol, dicalcium phosphate, sodium chloride, and alginic acid. Disintegrators commonly used in the formulations of this invention include croscarmellose, microcrystalline cellulose, corn starch, sodium starch glycolate and alginic acid.

Formulations suitable for topical administration may be presented as creams, gels, pastes, or foams, containing, in addition to the active ingredient, such carriers as are appropriate. In some embodiments the topical formulation contains one or more components selected from a structuring agent, a thickener or gelling agent, and an emollient or lubricant. Frequently employed structuring agents include long chain alcohols, such as stearyl alcohol, and glyceryl ethers or esters and oligo(ethylene oxide) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations may further include other components, e.g., astringents, fragrances, pigments, skin penetration enhancing agents, sunscreens (e.g., sunblocking agents), etc.

Particular formulations of the invention are in an oral liquid form. The liquid can be a solution or suspension and may be an oral solution or syrup, which is included in a bottle with a syringe graduated in terms of milligram amounts which will be obtained in a given volume of solution. The liquid solution makes it possible to adjust the volume of solution for appropriate dosing of small children, who can be administered fenfluramine in an amount anywhere from 1.25 mg to 30 mg and any amount between in 0.25 milligram, increments and thus administered in amounts of 1.25 mg, 1.5 mg, 1.75 mg, 2.0 mg, etc.

In alternate embodiments, the dispensing device may be a syringe or graduated pipette useful for delivering varying doses of the fenfluramine liquid. In another embodiment, the dispensing device is a metered dosing device capable of dispensing a fixed volume of fenfluramine liquid. In one exemplary embodiment, the dose delivered by the metered dosing device is adjustable.

The formulation may be a solution or suspension and is prepared such that a given volume of the formulation contains a known amount of active fenfluramine.

For example, in one embodiment of this aspect, the dispensing device is a syringe is graduated in one milliliter increments and the liquid fenfluramine formulation is characterized such that one milliliter in volume of formulation includes precisely one milligram of fenfluramine. In this manner, the patient may be correctly dosed with a desired milligram dosage of fenfluramine based on a volume of liquid formulation administered to the patient orally.

In alternate embodiments, the dispenser is a syringe connected to the container and configured to withdraw the liquid formulation from the container, wherein the syringe is marked with levels of graduation noting volume of formulation withdrawn, or a metered dose dispenser for delivering a predetermined volume of the formulation to said patient, or a metered dispensing device calibrated to deliver a predetermined volume of the liquid, permitting convenient, consistent, and accurate dosing.

In a method of the present invention, fenfluramine can be employed as a monotherapy in the treatment of epilepsy or epileptic encephalopathy. Alternatively, fenfluramine can be co-administered in combination with one or more pharmaceutically active agents, which may be provided together with the fenfluramine in a single dosage formulation, or separately, in one or more separate pharmaceutical dosage formulations. Where separate dosage formulations are used, the subject composition and ore or more additional agents can be administered concurrently, or at separately staggered times, i.e., sequentially.

A method of the present invention can be practiced on any appropriately diagnosed patient. In alternate exemplary embodiments of the present invention, the patient is aged about 18 or less, about 16 or less, about 14 or less, about 12 or less, about 10 or less, about 8 or less, about 6 or less or about 4 or less to about 0 months or more, about 1 month or more, about 2 months or more, about 4 months or more, about 6 months or more or about 1 year or more. Thus, in this embodiment, the diagnosed patient is about one month old to about 18 years old when treated.

The invention is further illustrated in the following Examples.

### Example 1

### Assessing Epileptic Patients for Risk of or Exhibiting Symptoms of Pulmonary Hypertension

### Inclusion and Exclusion Criteria

Epileptic patients may be recruited from childhood epilepsy clinics in Leuven and Antwerp, Belgium, and selected for inclusion in the study according to criteria comprising a combination of age, physical and psychological characteristics, and (optionally) resistance to treatment with conventional therapies. Details of selection criteria are provided below.

| | |
|---|---|
| MINIMUM | Eligible seizure types |
| REQUIREMENTS: | Tonic-clonic (GTC) - REQUIRED |
| Diagnosis with epilepsy or | Tonic (TS) |
| epileptic encephalopathy | Atonic (AS) |
| | Clearly recognizable focal (FS) |
| | At least 4 documented seizures during |
| | 4 week period prior to inclusion |
| | Minimum of 4 seizures in at least |
| | 2 separate weeks |
| Age | Between 3 and 18 years |

Once enrolled, subjects are removed from the study in cases of serious adverse events, non-compliance, or lack of efficacy. Treatment is also stopped in the event of increased severity and frequency of seizures after discussion with the principle investigator; cardiac abnormalities (specifically, valvular problems), and/or adverse events (specifically, SAE, SAR or SUSAR) after discussion with the principle investigator. Patients may also withdraw voluntarily. Upon withdrawing, a safety examination (i.e., blood sampling and cardiac ultrasound) is performed and fenfluramine use is tapered for one week at 50% of end dosage and then withdrawn completely.

During the study period, patients are prohibited from receiving certain medications and foods: (1) felbamate is prohibited as a concomitant medication unless the following criteria are met: the patient has been treated for at least 18 months prior to screening; has stable liver function and hematology laboratory tests, and the dose is expected to remain constant throughout the study; (2) drugs that interact with central serotonin, including imipramine, monoamine oxidase inhibitors, SSRIs, SNRIs, or vortioxetine; and (3) drugs or foods that potentially interact with fenfluramine via the CYP-2D6, CYPD-3A4, and/or CYP-2B6 pathways, except for pre-approved short-term use where required by medical necessity. Pregnancy testing, use of birth control, and breast feeding restrictions were also required during the study period and for the duration of subsequent fenfluramine treatment.

Study duration is 20 weeks, ending at week 20 after inclusion of 20 patients, i.e., 20 weeks following the enrollment of the 20^{th} patient. Patients who respond to treatment are enrolled in a follow-on study and assessed on an ongoing basis.

Patients are examined during six clinical visits (V1 through V6) scheduled at four week intervals, ending with V6 at week 20. Thereafter, responders continue in a follow-up study, with visits scheduled every three months. At each visit, endpoints and safety criteria are assessed, and dosages adjusted as necessary.

First, patients diagnosed with epilepsy or epileptic encephalopathy are selected.

Patients are then assessed for symptoms of pulmonary hypertension. A physician's assessment of epileptic patients for risk or exhibiting symptoms of pulmonary hypertension can include: physical examination, detailing a family history of cardiac and/or pulmonary issues, echocardiography, ventilation/perfusion scintigraphy, right heart catheterization, CT scan, etc.). The assessment may include tests to distinguish/diagnose the type of PH for which the subject is at risk, or is exhibiting symptoms.

An echocardiogram (echo) is a useful screening tool for assessing pulmonary arterial hypertension and is commonly used. It is also used for routine surveillance of patients once they have been diagnosed with pulmonary hypertension. It is frequently used because it is noninvasive, relatively inexpensive, and very accessible, being widely available, even in outpatient settings. The echocardiogram is an ultrasound of the heart using a computer with a transducer attachment. The transducer is then pushed against the patient's chest which emits sound waves that reflect when they encounter an object like a heart valve and the echo is measured and digitized allowing generation of a moving image on a display.

The output image allows a physician to examine the chambers of the right side of the heart for enlargement, thickening of the walls, and if the septum appears normal. Results can be used to estimate the pulmonary artery pressures using mathematical equations and the amount of blood being jetted back into the right atrium by the right ventricle. Findings from an echocardiogram can suggest a diagnosis of pulmonary hypertension. There are several findings from an echocardiogram that are suggestive of pulmonary hypertension: (i) an enlarged right atrium, (ii) an enlarged right ventricle with a thick wall, (iii) septal shift (meaning the septum of the heart has been pushed into the left side of the heart by the enlarged chambers of the right side of the heart), and (iv) calculated pulmonary artery pressures are elevated. Echo is not used to officially diagnose PAH: right heart catheterization is presently the only way to officially diagnose a patient as having pulmonary arterial hypertension.

Patients not at risk or not exhibiting symptoms of pulmonary hypertension are then treated with fenfluramine for treating symptoms of epilepsy or epileptic encephalopathy. Patients found to be at risk of or exhibiting symptoms of pulmonary hypertension are then treated with diet, exercise, medications, or any combination thereof for a period of one week, two weeks, three weeks, one month, two months, three months, six months, one year or two (or more) years.

After treatment for PH, patients are re-assessed for symptoms of pulmonary hypertension. Those patients who were at risk of, or were exhibiting symptoms of, pulmonary hypertension but did not develop PH after treatment and re-assessment for symptoms of PH are subsequently treated with fenfluramine in an amount effective to treat the symptoms of epilepsy or epileptic encephalopathy. Those patients who were exhibiting symptoms of PH, and after treatment and re-assessment are observed to have a reduction in or elimination of the PH symptoms are subsequently treated with fenfluramine in an amount effective to treat the symptoms of epilepsy or epileptic encephalopathy. Those patients exhibiting symptoms of PH who were treated but, upon re-assessment, the PH symptoms are found not to be reduced, ameliorated or eliminated are excluded from treatment with fenfluramine (as contraindicated).

All patients (pre- or post-PH treatment) not exhibiting symptoms of pulmonary hypertension are candidates for effective treatment of epilepsy or epileptic encephalopathy with fenfluramine or a pharmaceutically acceptable salt thereof.

At V1, inclusion and exclusion criteria are assessed, clinical diagnosis confirmed, and the following information is collected: baseline demographics, pre-baseline seizure counts, current treatment regimens (both AEDs and VNS), and sleep quality. In addition, safety blood samples are collected, blood levels of anti-epileptic drugs (“AEDs”) are determined, and cardiac function is evaluated using ultrasound imaging and EKG traces. Urine pregnancy tests in female subjects of childbearing potential are also performed, and the patient's quality of life is assessed by means of clinical global impressions (both parents and physician) and sleep quality. Patients meeting entry criteria are enrolled and begin a prospective baseline period.

Add-on treatment is begun at V2. Participants being receiving an initial fenfluramine dose of 0.2 mg/kg/day. Endpoints and safety criteria are assessed (seizure counting, adverse effects, pregnancy testing, and quality of life indicators (CGI, sleep scale)). Additional safety blood samples, AED blood levels and cardiac evaluation are done only if clinically indicated. A seizure diary and medication are dispensed.

At each of V3-V5, endpoints and safety criteria are again assessed. Dosages of non-responders are adjusted according to trial protocol.

At V6, endpoints and safety criteria are again assessed. Safety blood samples, AED blood levels and cardiac evaluation are repeated. Responders may enter the follow-up study. Non-responders begin FFA tapering, receiving 50% of the end dosage for 1 week and then discontinuing treatment.

Follow-up study: Responders at 20 weeks continue in a follow-up study, with visits scheduled at 3 month intervals. Patients receive a starting dose equal to the dosage received at week 20. At each visit, endpoints and safety criteria are assessed (seizure counting, current treatment, adverse effects, quality of life indicators (CGI, sleep scale)), pregnancy testing is performed, safety blood samples are collected, blood level AEDs are determined and cardiac function is evaluated using EKG and cardiac ultrasound. Dosages may be increased as necessary, up to a maximum of 30 mg/day (20 mg/day if stiripentol is a concomitant drug). Follow up ends when fenfluramine becomes available as a regular treatment or at the election of the patient and/or treating physician if serious side effects occur and/or the drug is no longer effective.

### Materials and Methods

Ethics and regulatory approvals: Trial conduct complies with the most recent version of the principles of the Declaration of Helsinki, the principles of GCP, and in accordance with all applicable regulatory requirements. The study protocol and related documents is subject to ethical review by all requisite authorities. Participants have given written informed consent prior to their enrollment and participation in compliance with all applicable laws, regulations and ethical guidelines as required, and ICFs are retained at participating trial sites in accordance with all applicable regulatory agencies and laws. All information and data related to the Study and disclosed to the Participating Site and/or Study Investigator are treated as confidential and will not be disclosed to third parties or used for any purpose other than the performance of the study. Data collection, processing and disclosure of personal data is subject to compliance with applicable personal data protections and personal data processing requirements.

Fenfluramine: Oral fenfluramine solution (2.5 mg/ml or 5 mg/ml) is provided by Zogenix Pharma. Starting dosage is 0.2 mg/kg/day BID; second step at 0.4 mg/kg/day BID; maximum dosage at 0.8 mg/kg/day BID or 30 mg/day BID, whichever is less. The drug is dispensed by Zogenix Pharma. Labeled bottles containing the oral fenfluramine suspension is given to patients and controlled at each visit. Bottle labels are kept in individual patient files. Calculation of bottle number and control of labels are done at the trial's conclusion. Patient compliance is assessed by control of oral solution quantity at each visit and collection of seizure diary with notification of drug intake.

Concomitant treatment: Lennox-Gastaut patients participating in the study receive concomitant treatment with two or more anti-epileptic drugs commonly used in the treatment of the disorder. The drug regimen is unchanged during baseline (the period from V1 to V2) and the full trial period (V2 to V6).

Laboratory tests: Blood analysis and urine pregnancy tests are done in central lab at UZ Leuven. Safety blood samples are tested for hemogram, electrolytes, liver function (SGOT, SGPT, LDH, PT) and kidney function (urea, creatinine)). AED blood level determination is limited to phenytoin, phenobarbital, carbamazepine, and valproate.

Safety assessment: Treatment safety is assessed using a combination of physical examination, blood testing, cardiac evaluation, and adverse event reporting. With respect to adverse event reporting, reporting is not required for expected AEs, moderate weight loss and decrease of appetite with no significant weight loss (<P3).

Data Handling and Statistical Analysis: Data is coded and is protected from disclosure outside of research teams according to the terms of the research protocol and the informed consent document. Subjects' names or other identifiers must be stored separately (“site file”) from their research data and replaced with a unique code to create a new identify for the subject. Coded data are not anonymous. Data is collected in standardized CRF.

A priori data on possible efficacy is unavailable. Sample size is set at 20. The study is not randomized. Descriptive analysis of outcome parameters is done at weeks 8, 12, 16 and 20. All included subjects are counted for analysis. Reasons for withdrawal are documented.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1.-16. (canceled)

17. A method of alleviating a symptom in a patient exhibiting symptoms of epilepsy or epileptic encephalopathy, comprising:
diagnosing the patient with epilepsy or epileptic encephalopathy;
assessing the patient as being at risk for or exhibiting symptoms of pulmonary hypertension;
treating the patient for symptoms of pulmonary hypertension;
re-assessing the patient for symptoms of pulmonary hypertension; and
administering to the patient treated, re-assessed and not exhibiting pulmonary hypertension a formulation of fenfluramine or a pharmaceutically acceptable salt thereof in an amount sufficient to alleviate a symptom of epilepsy or epileptic encephalopathy.

18. The method of claim 17, wherein the assessing comprises determining whether the patients have one or more risk factors or symptoms of pulmonary hypertension selected from the group consisting of:
risk factors such as primary pulmonary hypertension, chronic obstructive pulmonary disease, sleep apnea, heart failure, left ventricular failure, valvular heart disease;
initial symptoms such as exertional dyspnea, lethargy, fatigue; and late symptoms (exertional chest pain, angina, exertional syncope, peripheral edema, anorexia, abdominal pain).

19. The method of claim 17, wherein the assessing comprises:
obtaining an echocardiogram for the patient and examining the results for one or more signs of pulmonary hypertension selected from the group consisting of: (i) an enlarged right atrium, (ii) an enlarged right ventricle with a thick wall, (iii) a septal shift, and (iv) an elevated calculated pulmonary artery pressure.

20. The method of claim 17, wherein the assessing comprises:
measuring the patient's pulmonary artery pressures via right heart catheterization.

21. The method of claim 17, wherein the assessing determines that pulmonary artery systolic pressure is ≥28 mm of Hg or mean pulmonary artery pressure is ≥20 mm of Hg even after treating the patient for pulmonary hypertension.

22. The method of claim 17, wherein the assessing comprises genetically testing the selected patients diagnosed with epilepsy or epileptic encephalopathy and identifying genetic risk factors for pulmonary hypertension.

23. The method of claim 17, wherein the treating of patients for symptoms of pulmonary hypertension comprises administering diuretics (e.g., furosemide, spironolactone, amiloride) and/or vasodilators (e.g., epoprostenol, tadalafil, ambrisentan, sildenafil, iloprost, alprostadil, treprostinil).

24. The method of claim 22, wherein patients assessed and identified as having genetic risk factors for pulmonary hypertension are treated prophylactically with one or more medications selected from the group consisting of furosemide, spironolactone, amiloride, epoprostenol, tadalafil, ambrisentan, sildenafil, iloprost, alprostadil and treprostinil to mitigate the risk.

25. The method of claim 24, wherein the treating of patients at risk or exhibiting symptoms of pulmonary hypertension further comprise reducing sodium intake.

26. The method of claim 25, wherein the treating of patients at risk or exhibiting symptoms of pulmonary hypertension comprises a regimen of diet and increased exercise.

27. The method of claim 17, wherein the patient is diagnosed with a form of epilepsy selected from the group consisting of Dravet syndrome, Lennox-Gastaut syndrome, and epileptic encephalopathy.

28. The method of claim 17, wherein a symptom of the epileptic encephalopathy is seizure, and wherein the fenfluramine is formulated with a pharmaceutically acceptable carrier and administered in dose less than 10.0 mg/kg/day to 0.01 mg/kg/day.

29. The method of claim 17, wherein the patient is diagnosed with Dravet syndrome.

30. A kit, comprising:
a container comprising a plurality of doses of a formulation comprising a pharmaceutically acceptable carrier and an active ingredient comprising fenfluramine; and
instructions for treating the patient diagnosed with epilepsy or epileptic encephalopathy, wherein the instructions warn against administering the formulation to the patient if the patient is exhibiting signs of pulmonary hypertension.

31. A method of identifying a patient population as candidates for effective treatment of symptoms of epilepsy or epileptic encephalopathy with fenfluramine or a pharmaceutically acceptable salt thereof, comprising:
selecting patients diagnosed with epilepsy or epileptic encephalopathy;
assessing the patients for risk or exhibiting symptoms of pulmonary hypertension;
treating patients at risk or exhibiting symptoms of pulmonary hypertension;
re-assessing the patients for symptoms of pulmonary hypertension; and
identifying treated and re-assessed patients not exhibiting pulmonary hypertension as a population of candidates for effective treatment of epilepsy or epileptic encephalopathy with fenfluramine or a pharmaceutically acceptable salt thereof.

32. The method of claim 31, wherein the patient is diagnosed with a form of epilepsy selected from the group consisting of Dravet syndrome, Lennox-Gastaut syndrome, and epileptic encephalopathy.

33. The method of claim 31, wherein the patient is diagnosed with Dravet syndrome.

34. The method of claim 31, wherein the assessing comprises genetically testing the selected patients diagnosed with epilepsy or epileptic encephalopathy and identifying genetic risk factors for pulmonary hypertension.

35. The method of claim 34, wherein patients assessed and identified as having genetic risk factors for pulmonary hypertension are treated prophylactically with one or more medications selected from the group consisting of furosemide, spironolactone, amiloride, epoprostenol, tadalafil, ambrisentan, sildenafil, iloprost, alprostadil and treprostinil to mitigate the risk.

36. The method of claim 31, wherein the assessing comprises:
measuring the patient's pulmonary artery pressures via right heart catheterization.
